(19) United States
(12) Patent Application Publication
(10) Pub. No.: US 2012/0121712 A1
(43) Pub. Date: May 17, 2012

(54) CELLULAR ELECTRIC STIMULATION MEDIATED BY PIEZOELECTRIC NANOTUBES

(76) Inventors: Gianni Ciofani, Monterosso al Mare (La Spezia) (IT); Vittoria Raffa, Calcinaia (Pisa) (IT); Serena Danti, Pisa (IT); Arianna Menciassi, Pontedera (Pisa) (IT); Paolo Dario, Livorno (IT); Mario Petrini, Pisa (IT); Alfred Cuschieri, St. Andrews (GB)

(21) Appl. No.: 13/264,158

(22) PCT Filed: Apr. 14, 2010
(86) PCT No.: PCT/IB10/51602

§ 371 Date: Jan. 24, 2012

(30) Foreign Application Priority Data
Apr. 14, 2009 (IT) FI2009A000076

(51) Int. Cl. A61N 1/36; A61K 9/00; C12N 5/071; C12N 13/00; A61K 33/22; A61K 9/50; B82Y 5/00

(52) U.S. Cl. 424/490; 424/657; 424/400; 435/395; 435/173.1; 607/61; 977/837; 977/925

(57) ABSTRACT

Piezoelectric nanotransducers for use in an in vivo treatment of cell stimulation through electrical stimulation are described. The nanotransducers are localized in a target site, and an electrical stimulus is induced in the same site through external stimulation of the nanotransducers by ultrasonic waves.

### TECHNICAL FIELD OF THE INVENTION

The invention relates to a method for inducing non-invasive electrical cell stimulation, both in vitro and in vivo, by use of piezoelectric nanovectors. Specifically, these are boron nitride nanotubes (BNNTs) capable of converting a specific non-invasive external stimulus (ultrasonic waves) into electrical inputs able to stimulate cells.

### STATE OF THE PRIOR ART

Electrotherapy

Electrical cell stimulation finds numberless applications in the biomedical field, such as deep brain stimulation, gastric stimulation following gastroparesis, cardiac stimulation, muscle stimulation, etc. In particular, in neurological disorders electrical brain stimulation is often the sole form of therapy. It has long been demonstrated that appropriate electrical stimulations induce a positive response in cultured cells with regard to proliferation, metabolism or production of specific substances. Supronowicz and collaborators demonstrate that electrical stimulation in the presence of carbon nanotubes improves the proliferation and the production of extracellular material of osteoblasts in vitro stimulated by electric current impulses (Supronowicz et al. (2002) Journal of Biomedical Materials Research, 59, p. 499-506). Chachques et al. (2004) International Journal of Cardiology, 95, p. 68-69 indicate how electrical stimulation in vitro of myocardial stem cells increases their proliferation, development, organization in myotubes and differentiation.

Deep brain stimulation is a treatment of proven effectiveness for high-impact pathologies such as Parkinson's disease, chronic tremor, dystonia and other hyperkinetic disorders. All internationally accepted clinical applications of functional electrical stimulation are based on a direct excitation of nervous structures and—in the case of muscle functions—on an indirect activation of the muscle.

Moreover, it has been demonstrated, in a rat study, that electrical stimulation is capable of re-establishing the electrical and electrochemical properties of muscle membrane even after various degrees of degeneration, not merely once, but even repeatedly.

Another rat study (Carraro et al. (2002) Basic and Applied Myology, 12, p. 53-63) demonstrated a low but lasting regenerative ability at cell level (regenerative myogenesis) in untreated denervated muscle, and, in addition, a substantial increase of this activity after repeated muscle lesions. Alike myogenic stimuli were observed in paraplegic patients with peripheral denervation of lower limbs.

However, current procedures for performing electrical stimulation are highly invasive. The procedure for performing a brain stimulation envisages inserting intracerebral electrodes and applying an implantable impulse generator to be connected to the electrodes themselves. This in vivo stimulation entails a large number of contraindications. Among them, uncontrollable coagulopathy and the possible risk of generating post-surgical dementia, with the entailed psychopathologies, have to be mentioned. In addition, the risk of an onset of ventriculomegaly, of subdural, subarachnoid, intraventricular or intracerebral hematoma is non-negligible. Last but not least, the risk of hemorrhages, even serious ones, reaches the neighborhood of 3-5% for each patient, while cases of strokes, infections and cerebral lesions are not absent. All of these episodes can lead to long-term disabilities and, in the worst cases, to patient's death. Moreover, oft-times infections caused by devices, not responding to antibiotic treatments, lead to a definitive removal of the electrodes.

Owing to all these complications, it is easy to understand how to date nervous stimulation, though effective and promising, is restricted to the sole treatment of advanced stages of the pathologies, when every other pharmacological therapy proves totally ineffective.

Also testing on muscle tissue demonstrated functional electrical stimulation to be an effective and powerful instrument for maintaining, functionally recovering and reconstructing denervated musculature. However, the technique entails the same high-invasivity problems already found in the case of nervous stimulation.

In human therapy and diagnostics the use of nanostructures such as nanoparticles, nanotubes, nanofibrils, is known.

Pat Appln. EP-A-1593406 (M. Pizzi et al.) describes a device for electrochemotherapy comprised of micro- or nano-capacitors made of composite pyroelectric or piezoelectric material and a medicament. The device may be injected in the circulation and activated from the outside, in order to release the medicament. The micro/nano-capacitor is activated by a source of vibrations or electromagnetic radiations. In said document no reference is made to cell stimulation, nor to nanotubes, or to ultrasonic waves as external source.

Pat. Appln. EP-A-1818046 (M. Pizzi et al.) describes a micro-device comprised of a nano-capacitor made of ferroelectric, pyroelectric or piezoelectric material enclosed by a membrane and containing a drug. The device can be injected into the bloodstream, and from outside, with appropriate stimulation, it is possible to generate a potential difference which porates the membrane (electroporation) and releases the drug. Both the aims and the design of the device depart from the object of the present invention.

Pat. Appln. US 2009022655 describes boron nitride nanotubes for cancer treatment by BNCT (Boron Neutron-Capture Therapy). The document also describes the use of carbon nanotubes as vectors for anti-tumor medicaments. In an embodiment of the invention, carbon nanotubes containing the medicament are exploded via high-power ultrasonic waves. This document does not describe cell stimulation, nor an applying of the piezoelectric effect of the boron nitride nanotubes described therein.

Object of the present invention is to provide novel instruments and techniques allowing the applying of electrical cell stimulation without incurring in the severe adverse effects typical of present-day electrotherapy techniques.

### SUMMARY OF THE INVENTION

The present invention is based on the surprising discovery that piezoelectric nanotransducers can be effectively employed in a completely non-invasive treatment of electrotherapy, in which the electrical stimulus generated by the nanotransducers is caused through a (wireless-type) stress external to the patient's body, by ultrasonic waves of appropriate power. Therefore, the present invention is based on the experimental demonstration that not only piezoelectric nanotransducers can be stimulated by an ultrasonic field generated externally to the system in which the same have been localized, but the electrical stimulus produced by the nanotransducer localized inside the target cell is sufficiently high to cause an effective electrical stimulation in a real cell system, in vitro or in vivo.

Therefore, a first object of the present invention are piezoelectric nanotransducers for use in an in vivo treatment of cell stimulation through electrical stimulation comprising the following steps: localizing the nanotransducers in a target site; inducing an electrical stimulus in the same site through external stimulation of nanotransducers by ultrasonic waves.

In an embodiment of the invention the piezoelectric nanotransducers are made biocompatible by coating with pharmaceutically acceptable polymers and/or functionalized with specific ligands having affinity for the target site and/or functionalized with marker molecules that allow tracking thereof.

In a specific embodiment of the invention the piezoelectric nanotransducers are nanotubes, e.g. boron nitride nanotubes.

The nanotransducers of the invention are utilized in a regenerative or reconstructive treatment of various tissues via internalization in tissue cells and their subsequent electrical stimulation.

A second object of the invention is a preparation for pharmaceutical use and a method for preparing, comprising piezoelectric nanotubes capable of being stimulated by an ultrasonic remote field and a pharmaceutically acceptable excipient for use in an electrotherapy treatment; in particular, a formulation in liquid form of suspension/solution comprising said nanotubes in a non-aggregated form and a biocompatible polymer as dispersing agent.

A third object of the invention is represented by a method in vitro for electrical cell stimulation (cell stimulation via electrical stimulation) comprising the following steps:

dispersing the piezoelectric nanotransducers in culture medium or cell growth supports,

incubating the cells in said culture medium or growth support,

inducing an electrical stimulus through a stimulation of nanotransducers by an external ultrasonic field. In an embodiment of this object the growth supports are polymeric scaffolds for tissue engineering or implant or adhesion substrates.

Further objects of the invention are supports (scaffolds) for cell growth or cell adhesion substrates or for use in tissue engineering, in vitro or in vivo, comprising the piezoelectric nanotransducers as described above, capable of producing an electrical stimulus as a result of external stimulation with ultrasounds (US).

The solution proposed by the invention offers the advantage of inducing an effective electrical stimulation maximizing the benefits of electrical cell stimulation, but eliminating or drastically reducing adverse problems and side effects caused by present-day clinical technologies. The proposed method totally reduces the invasiveness of present-day procedures for electrical stimulation of tissues in vivo and remarkably simplifies any form of electrical stimulation in vitro. With regard to in vitro cell stimulation, the proposed solution abolishes the electrical circuits for stimulation, electrical connections or other devices connected to the cultures, thereby facilitating the system for the improvement of cell growth conditions. The nanotransducers may be dispersed in the culture medium (CM) or embedded into support structures for cell growth (polymeric scaffolds, adhesion substrates, etc.) and then stimulated through ultrasonic fields. Moreover, both in in vitro and in vivo applications, the powers involved can be modulated casewise in order to better adapt them to different needs.

### DESCRIPTION OF THE FIGURES

FIG. 1: a schematic cell model illustrating the present invention is reported. Every cell internalizing BNNTs is subject to an internal electrical stimulus as a consequence of an external ultrasound stimulus.

FIG. 2: results of MTT assay after 24, 48, 72 of primary human osteoblasts (HOBs) incubation with 0 (control), 5, 10, 15 μg/ml of BNNTs (n=6) are illustrated. No statistically significant difference was observed among groups.

FIG. 3: results of a cell internalization test are reported. BNNTs labeled with fluorescent markers (quantum dots) were detected inside cells by fluorescence microscopy after 6 h of HOBs incubation with BNNTs—containing CM.

FIG. 4: TEM micrographs of cytoplasm sections of HOBs or of controls, or of HOBs treated with BNNTs are shown. Results confirm internalization of BNNTs and show the presence of nanoparticles compatible with BNNTs in the cytoplasmatic vesicles. Internalization of BNNTs occurs by endocytosis.

FIG. 5: RT-PCR analysis results are reported, and gene expression of HOBs treated both with a single stimulation (either BNNTs or US) and a combined one (BNNTs+US) is shown. Runx2 expression was found to be downregulated by BNNTs, whereas OPN expression levels were enhanced by US. Coll I expression did not vary, whereas AP and OCN expressions were synergistically influenced by treatments with both BNNTs and US.

FIG. 6: OCN production levels per cell are reported. Samples treated with BNNTs and with BNNTs+US exhibited higher OCN production with respect to US-treated samples and to controls.

FIG. 7: results of colorimetric cytochemical analysis according to von Kossa are reported; analysis was related to production of calcium salts (black) on samples of primary human osteoblasts untreated (HOBs), or stimulated with ultrasounds (HOBs+US), or treated with nanotubes (HOBs+BNNTs) or treated with nanotubes and stimulated with ultrasounds (HOBs+BNNTs+US). The highest calcification (darker staining) was attained in HOBs treated with BNNTs and stimulated with US.

FIG. 8: fluorescence images of human glioblastoma multiforme cells incubated for 90 min with 10 μg/ml fluorescent BNNTs (conjugated with quantum dots) functionalized (a) or non-functionalized (b) with folic acid are reported.

FIG. 9: images of calcein-labeled PC12 cells after 5 days of treatment as described in Example 14. FIG. 9a: cells not incubated with GC-BNNT and not treated with ultrasounds; FIG. 9b: cells incubated with GC-BNNT and not treated with ultrasounds; FIG. 9c: cells not incubated with GC-BNNT and treated with ultrasounds; FIG. 9d: cells incubated with GC-BNNT and treated with ultrasounds.

FIG. 10: PC12 cells treated as described in Example 14. FIG. 10a: analysis of differentiation tendency; FIG. 10b: number of neurites per cells; FIG. 10c: neurite length.

### DETAILED DESCRIPTION OF THE INVENTION

Nanotransducers

Piezoelectric nanotransducers suitable for the present invention are nanostructures known per se, such as particles, tubes, rods, spheres, fibrils, filaments having at least one dimension, preferably two or three, below 100 nm and consisting of or comprising a piezoelectric material. An example of a useful material is boron nitride; other examples of useful materials include, e.g., barium titanate, strontium titanate (in general, all perovskites) and polyvinylidene fluoride (PVDF). The nanotube group comprises single-walled, double-walled or multi-walled nanotubes, and they can be open on the two ends as well as on one end only, or closed on the two ends. An example of such nanotubes are boron nitride nanotubes.

Boron nitride nanotubes (BNNTs) are structurally analogous to the more famous carbon nanotubes (CNTs): alternating B and N atoms entirely substitute for C atoms in the classic shape of a rolled-up graphite sheet, without practically any change in interatomic distances. BNNTs are produced through a ball-milling atomization process followed by annealing as described by Chen Y. et al. (1999) Chemical Physics Letter, 299, p. 260-264 or by Yu J. et al. (2005) Chemistry of Materials, 17, p. 5172-5176. In the international scientific community they are sparking off a remarkable surge of interest (Chopra et al. (1995) Science, 269, p. 966-967) and have attracted wide attention owing to their unique and relevant physico-chemical properties, making them ideal candidates for several structural and electronic applications (Terrones et al., (2007) Materials Today, 10, p. 30-38.

In addition to a high Young's modulus (Chopra et al. (1998) Solid State Communications 105, p. 297-300), similar to that of CNTs, BNNTs own superior chemical and thermal stabilities. Compared to CNTs, BNNTs exhibit stabler electrical properties, with an uniform band gap of 5.5 eV, unlike CNTs which exhibit diversified electrical behaviors, ranging from those typical of semiconductors to those of excellent conductors. In fact, the progress toward controlling CNTs chirality (and therefore their electrical properties) is modest, whereas BNNTs exhibit a structure preferably defined as “zigzag” due to the polar nature of the B—N bond. All these properties make BNNTs particularly interesting for a number of nanotechnological applications. BNNTs own excellent piezoelectric properties. Piezoelectricity is the ability of some crystals to generate an electric potential difference in response to applied mechanical stress. Ab initio calculations of the spontaneous polarization and piezoelectric properties of BNNTs have demonstrated that they function as excellent piezoelectric systems with response values larger than those of most piezoelectric polymers, and comparable to those exhibited by wurtzite-based semiconductors. In addition, BNNT bending forces have been measured directly inside high resolution transmission electron microscopy (HRTEM), confirming an exceptional flexibility of these structures (Golberq et al. (2007) Advanced Materials, 19, p. 2413-2432). These observations underpin the remarkable potential of BNNTs as efficient and innovative nanovectors.

Biocompatible Nanotransducers

The first requirement for biomedical applications is the production of suspensions, stable in physiological solutions and biocompatible, of nanotransducers that may be administered without causing immune reactions and that be readily internalized into the cells of interest. A highly promising approach envisages the use of polymers coating the nanostructure and making it biocompatible and easily dispersable or quasi-soluble in aqueous means. Polymers suitable for this purpose are those such as polysaccharides, e.g. chitosan, glycol chitosan, poly-L-Lysine (PLL), polyethylene imine (PEI), polylactic, polyglycolic, polyaspartic acid or copolymers thereof. Preferably, the polymer is a cationic polymer such as polylysine and polyethylene imine. Methods for the polymeric, covalent or non-covalent coating of nanotubes with positively charged polymers such as polyethylene imine are described by Ciofani et al. (2008) J. Nanosci. Nanotechnol, 8, p. 6223-6231, or in Ciofani et al. (2008) Biotechnology and Bioengineering, 101, p. 850-858. Coating methods with polylysine are described hereinafter in the examples.

The use of the above-indicated polymers, beside making the nanotransducer biocompatible, allows to obtain dispersions that are homogeneous, aggregate-free and therefore easily internalizable in the target cell.

Moreover, the nanotransducers according to the invention may be functionalized with various types of molecules, first of all with marker molecules capable of being detected, ensuring their tracking up to inside the target cell.

Any type of known marker suitable for cell assays may be used for this purpose: for instance fluorescent substances, chromophores or radioactive isotopes. The nanotransducers may then be functionalized with specific ligands for therapeutic or diagnostic targeting to cells of interest. These ligands can be specific antibodies or fragments thereof, for instance IgG, ligands specific for particular membrane receptors, e.g. folic acid, or other known biopartners. It has recently been demonstrated how BNNTs functionalized with folic acid are preferably internalized by glioblastoma cells (FIG. 8) overexpressing the receptor for said substance (Ciofani et al. (2009) Nanoscale Res Lett., 4, p. 113-121).

Functionalization with specific molecules has a particular usefulness in vivo, and allows vector recognition by target cells. Targeting effectiveness of specific cells is essential in vivo, e.g. in applications of nervous or muscle stimulation: e.g., a dispersion of functionalized BNNTs injected in the bloodstream is localized at the site where electrical stimulation is required, the latter being then carried out through application of localized external ultrasonic fields.

Nanotransducers Localization/Administration

The piezoelectric nanovectors according to the invention are localized in the target site. This occurs by internalization of nanotransducers in the cells of the site as a result of direct administration into the target site, e.g. through injection in situ in the tissue to be treated. An alternative and less invasive administration pathway is the administration into the bloodstream of nanovectors functionalized with specific ligands that, thanks to their affinity, be capable of carrying the nanostructures and of accumulating them at the target site, enabling their internalization by cells of interest.

A further administration option consists in the encapsulation of BNNTs in lipid microbubbles such as those employed as contrast agent (e.g., SonoVue, a product for clinical use). These phospholipid microbubbles contain sulphur hexafluoride SF₆ (a completely harmless and scarcely soluble gas, eliminated at the pulmonary level), enter the bloodstream by injection of a suspension, having a size comparable to red cells (2-5 μm); then arrive into the capillaries, but do not exit the bloodstream. Said microbubbles can incorporate the BNNTs and carry them to the site of interest, where the former are exploded by ultrasonic stimulus and free the latter; BNNTs, on the contrary, can exit the microcirculation and reach the target site under ecographic monitoring. Said microbubbles can further be employed as possible drug-carriers for targeted chemotherapy.

Exemplary tissues susceptible of being treated in accordance with the present invention are the muscle, nervous, bone, cartilaginous, myocardial tissues, the tissues comprising all sensory cells, such as inner ear hair cells, rods and cones of the retina, cells of taste, touch and smell, i.e., all those cells that own chemo-, thermo-, photo-, mechanoreceptors and transform a received stimulus into a difference in membrane polarization which activates the neighboring neuron, or any other tissue or organ, such as tendons and ligaments, requiring a regenerative or reconstructive treatment or an acute, chronic, neuromuscular pain treatment, or a healing treatment of damaged tissues.

Specific cell types whose growth is activated, stimulated or promoted by electrical stimulation with piezoelectric nanotransducers comprise muscle cells, myoblasts, neural cells, myocardial cells, osteoblasts, osteoclasts, cardiac stem cells, stem cells in general and the sensory cells mentioned in the foregoing.

By way of example, in case of stimulation at the level of the nervous system, a BNNTs suspension can be injected in situ or into the bloodstream upon appropriate functionalization and then, thanks to an external stimulation, power generation can be attained with no need of highly invasive transcutaneous and penetrating implants.

Method In Vitro and Supports for Cell Growth

In an alternative embodiment of the invention, the piezoelectric nanovectors are utilized in a method in vitro for cell activation, stimulation or growth promotion and/or regeneration through electrical stimulation.

With regard to in vitro stimulation and tissue engineering applications, the present invention facilitates cell stimulation and the possibility of improving the conditions of cultured tissues in terms of metabolism, proliferation, extracellular matrix production and metabolite production. In fact, on several cell typologies electrical stimulation has long been proved to have positive effects on their growth. The solution represented by the invention allows to achieve these results with no need of electrical circuits for stimulation, electrical connections or other devices connected to the cultures. Moreover, the proposed nanotransducers can both be administered in the culture medium, as described, and embedded in support structures for cell growth such as polymeric scaffolds, or adhesion substrates, etc., and then stimulated by ultrasonic fields as described below.

In case of cultures in a liquid medium, the piezoelectric nanotransducers, preferably made biocompatible and/or functionalized with specific ligands or with marker molecules as described above, are stably and homogeneously dispersed in the culture medium, in concentrations not entailing toxic effects for the cultured cell. Concentrations comprised between 5 and 100 μg/ml, e.g. concentrations of 5, 10, 15, 25, 50, 75 μg/ml yielded no toxic effect whatsoever after incubation of up to 72 h.

Fluorescence assays have also highlighted that incubations ranging from 1 to 10 h, for instance 1, 3, 5, 6 h, depending on cell type, are sufficient to obtain internalization of nanotransducers in the cell. A 6-h incubation proved effective to internalize boron nitride nanotubes in human osteoblasts.

In case of cultures on solid supports, e.g. polymeric ones, or on semisolid supports, e.g. gels, the piezoelectric nanotransducers are embedded in homogeneous form in the support during the preparing thereof. In particular, the method for preparing supports envisages a step in which the piezoelectric nanotransducers are dispersed in a solution or dispersion or emulsion containing the polymer or its monomers, a step in which the monomers are polymerized and a step in which the liquid medium is removed with obtainment of a solid or semi-solid matrix containing the nanotransducers. Supports for cell growth are known per se. The polymers utilized for preparing them are biocompatible and cytocompatible polymers. In particular, the polymers utilized in tissue engineering for in vitro production of tissues and their subsequent implanting in vivo should moreover be provided with the following properties: bioabsorbable, (or biodegradable or bioerodible), immunologically inert, non non-toxic, non-carcinogenic.

Known polymers useful for preparing growth supports are, for instance, polylactate, polyglycolate, copolymers thereof, polypyrrolidone, polymers derived from cellulose, chitosan/chitin, polylysine, polyethylene imine. Other polymers suitable for preparing the supports of the invention are described in WO-A-2001/087193, whose content is incorporated in the present application. A method for preparing supports according to any one of the claims 13 to 15, comprising the following steps:

dispersing the piezoelectric nanotransducers into a solution or dispersion or emulsion containing the polymer or its monomers,

removing the liquid medium with obtainment of a solid or semi-solid matrix containing the nanotransducers.

Internalization in the Target Cell.

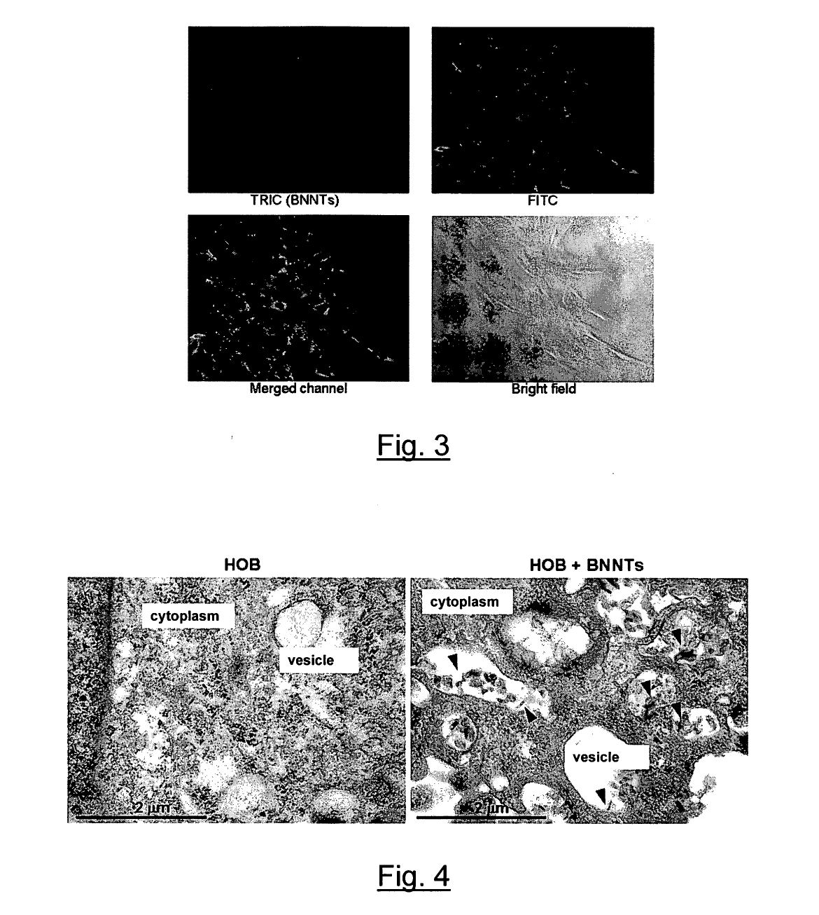

Both when operating in vivo and in vitro, the effectiveness of the cell stimulation treatment depends on the level of internalization of piezoelectric nanotransducers in the cell of interest. Fluorescence tests have demonstrated that incubation times ranging between 1 and 10 h are sufficient to attain an effective internalization of nanotransducers of the invention. For instance, human glioblastoma multiforme cells incubated for 90 min with 10 μg/ml fluorescent BNNTs functionalized with folic acid demonstrated high internalization levels (FIG. 8). Also cultures of primary human osteoblasts effectively internalized boron nitride nanotubes treated with poly-L-lysine and labeled with fluorescent markers after a 6-h incubation (FIG. 3).

Also cultures of nervous PC12 cells effectively internalized glycol-chitosan treated boron nitride nanotubes after a 12-h incubation.

Ultrasonic waves are widely utilized in several fields of medicine, owing to their low invasiveness and practically total absence of side effects. Among known main applications, there should be mentioned diagnostics (echographic examination), post-traumatic pain treatment, applications in rehabilitation, aesthetic medicine, etc.

In accordance with the present invention, once localized in vivo in the target site and internalized by the cells of interest, or when dispersed in culture media or embedded in adhesion substrates or polymeric supports (scaffolds) for cell growth, the piezoelectric nanotransducers are stimulated by a field of ultrasonic waves, which are in fact mechanical sound waves. These are produced by a generator external to the in vitro cell system, or external to the body of the patient undergoing treatment. In in vivo treatments the field is usually located near the target site.

To generate ultrasonic waves suitable for present invention, there may be used any one commercial device allowing adjustment of signal frequency and voltage, therefore of signal strength. E.g., a standard apparatus with ecographic stimulation heads and adjustable power and frequency may be used.

Merely by way of example, hereinafter a model of the piezoelectric behaviour of a nanovector (nanotube) is described. Piezoelectricity, as already mentioned hereto, is the combination of the electric behaviour of the material and Hooke's law; such a combination may be summarized by the following equation

\vec{D}=∈₀∈ᵣ\vec{E}+4π\vec{P} (1)

where D is the overall polarization of the material, E is the electric field, ∈₀ is the dielectric constant of vacuum, ∈ᵣ is the relative dielectric constant and P is the polarization due to piezoelectric phenomena, expressed by

\vec{P}= d̅\vec{σ} (2)

where d is a 3×6 matrix of the piezoelectric constants and σ is the stress tensor simplified to 6 components. In the absence of charges inside the material, from Maxwell's equations it is obtained

∇\vec{D}=∈₀∈ᵣ∇\vec{E}+4π∇\vec{P}=0 (3)

and therefore the following system:

$$\frac{\partial {E}_{x}}{\partial x} = - \frac{4 π}{{ɛ}_{0} {ɛ}_{r}} \frac{\partial {P}_{x}}{\partial x} \\ \frac{\partial {E}_{y}}{\partial y} = - \frac{4 π}{{ɛ}_{0} {ɛ}_{r}} \frac{\partial {P}_{y}}{\partial y} \\ \frac{\partial {E}_{z}}{\partial z} = - \frac{4 π}{{ɛ}_{0} {ɛ}_{r}} \frac{\partial {P}_{z}}{\partial z} \qquad ( 4 )$$

For simplicity's sake, let us assume that the nanotube, of length l, be subjected, by effect of an ultrasonic wave, to a stress σ_{zz} along its vertical axis z. The sole non-nil component of P will be

P_{z}=d_{zzz}σ_{zz} (5)

from which it is deduced

$${E}_{z} = - \frac{4 π}{{ɛ}_{0} {ɛ}_{r}} {d}_{zzz} {\int }_{- \frac{l}{2}}^{\frac{l}{2}} \frac{\partial {σ}_{zz}}{\partial z} d z = - \frac{4 π}{{ɛ}_{0} {ɛ}_{r}} {σ}_{zz} {d}_{zzz} \qquad ( 6 )$$

by integrating E_{z} along axis z, we obtain

$$Δ V = - {\int }_{- \frac{l}{2}}^{\frac{l}{2}} {E}_{z} d z = \frac{4 π}{{ɛ}_{0} {ɛ}_{r}} {σ}_{zz} {d}_{zzz} l \qquad ( 7 )$$

which represents the potential difference at the ends of the nanotube generated by application of the mechanical stress σ_{zz}. Of course, the control parameters of this stress (in our case the ultrasound source, the frequency, number, duration and strength of the impulses) vary depending on the applications. Optimum conditions for obtaining effective results for every cell system are easily obtainable empirically by any person skilled in the art.

While any frequency ranging from 20 kHz to 20 MHz may be usefully employed in the methods of the invention, the strength of the ultrasonic signal must remain below the critical threshold of damage to irradiated cells and tissues. This threshold varies if the method is applied in vitro or in vivo, and strongly depends on application times. In the methods of the invention, signal application times range from 5 to 30 s, repeated two, three or more times per day and per week.

For said application times, signal strength may range between 50 mW/cm² and 25 W/cm². Preferably, an in vivo treatment involves strengths of between 100 mW/cm² and 10 W/cm² for an application time of ≦30 sec in the case of maximum strength. The in vitro treatment allows higher strengths, ranging between 10 W/cm² and 25 W/cm² always for applications of from 5 to 30 s repeated as indicated above. If a strength of 20 W/cm² is adopted, for application times of from 5 to 30 s it will develop an energy equal to 100-600 J/cm².

The effectiveness of US wave-induced cell electrotherapy techniques can easily be assessed by analyzing various cell parameters, generally recognized as indexes of cell development, differentiation, maturation or vitality.

An effective test consists in the assessment of cell expression levels of typical genes via techniques well-known to a person skilled in the art: PCR or RT-PCR or any other known assay.

A second test is the determination, e.g. through enzymatic, immunoenzymatic, immunoradiometric or colorimetric assays, of proteins expressed by the cell or of metabolites or any other organic or inorganic substance produced by the cell, whose levels may be correlated to the degree of activation or of cell vitality itself.

Electrophysiological tests usually employed for the study of cell membrane potential (under patch clamp, voltage clamp, current clamp regimen etc.) are particularly useful to verify interferences that nanotube-mediated stimulations induce on the potentials themselves and on electric signal propagation, in particular in neural networks.

Applications

Non-invasive electrical stimulation of cells can find numberless applications in the biomedical field, both clinical and pre-clinical, such as deep brain stimulation, gastric stimulation following gastroparesis, cardiac stimulation, muscle stimulation. With regard to clinical applications, deep brain stimulation is a treatment of proven effectiveness for high-impact pathologies such as Parkinson's disease, chronic tremor, dystonia and other hyperkinetic disorders.

In addition, cell stimulation finds wide use in regenerative medicine and/or tissue engineering applications. This technique has high potential for use as a novel method for rehabilitation of patients having muscle denervations of various origin. As to tissue engineering and regenerative medicine applications, the possibility of integrating BNNTs in polymeric substrates or scaffolds suitable for cell growth should be considered.

Moreover, this non-invasive method allows to improve the conditions of cultivated tissues in terms of metabolism, proliferation and production of extracellular matrix.

Disclaimer

Any element specifically identified in the present application is understood to be exemplary and non-limiting, therefore it may be excluded from the given protective scope without altering the gist of the invention.

The invention will hereinafter be illustrated by means of experimental examples.

### EXAMPLES

### Example 1

### Human Osteoblasts (HOBs) Isolation and Expansion

Trabecular bone samples, removed from the femoral head of a patient undergoing femoral joint replacement surgery, were used after obtaining informed consent. Samples were sectioned, under sterile conditions, into smaller pieces. Thereafter, bone fragments were placed in a sterile saline supplemented with antibiotics and antimycotics and washed several times in order to remove fat, marrow, tissue residuals and blood cells. Isolation was performed in accordance to the established method (Di Silvio et al. Human cell culture. London (UK): Kluwer Academic Publishers; 2001. p. 221-241). Cell migration from native tissue was observed within 1-2 weeks, leading to formation of an osteoid layer in the neighbourhood of the explant. Cells were cultured in a culture medium (CM) containing: DMEM low glucose (Sigma-Aldrich, Milan, I), 10% FCS (Invitrogen), 10% L-glutamine (Sigma-Aldrich), HEPES (Sigma-Aldrich), non-essential amino acids (Sigma-Aldrich), ascorbic acid (Sigma-Aldrich), antibiotics and antimycotics with no supplemental mineral. Upon reaching confluence, cells were passed 1:3. P1 cells were used for characterization via cytochemistry and immunohistochemistry. P2 human osteoblasts (HOBs) were employed for studies with BNNTs.

### Example 2

### BNNTs Preparation and Conjugation

BNNTs supplied by Australian National University, Canberra, Australia, were produced by using ball-milling and annealing method (Chen Y et al. (1999) Chemical Physics Letter 299, p. 260-264; Yu J et al. (2005) Chemistry of Materials 17, p. 5172-5176). Details relating to sample purity and composition (provided by the supplier) were: yield >80%, boron nitride >97 wt %, metallic catalysts (Fe and Cr) derived from the milling process ˜1.5 wt % and adsorbed O₂ ˜1.5 wt %.

The polymer used for the aqueous suspension and dispersion of BNNTs was poly-L-lysine (PLL) obtained from Fluka (81339), molecular weight 70,000-150,000. All experiments were carried out in phosphate buffered solution (PBS) as described previously (Ciofani G. et al. (2008) Biotechnol. Bioeng. 101, p. 850-858). Briefly, samples of BNNT powder in a 0.1% PLL solution were ultrasonicated for 12 h with a Branson sonicator 2510 (Bransonic). The output power of the sonicator was set at 20 W for all experiments. Next, the samples were centrifuged at 1,100×g for 10 min to remove nondispersed residuals and impurities.

Excess PLL was removed by ultracentrifugation, three cycles at 30,000×g for 30 min at 4° C. (Allegra 64R, Beckman). PLL-BNNT dispersion was obtained as a result of the noncovalent coating of the nanotubes with PLL. Spectrophotometric analysis was carried out with a LIBRA S12 Spectrophotometer UV/Vis/NIR (Biochrom) to characterize the dispersions and to quantify BNNTs concentrations (Ciofani et al. (2008) J. Nanosci. Nanotechnol. 8, p. 6223-6231).

PLL-BNNTs were covalently bound with quantum dots functionalized with carboxyl groups for localization/cellular tracking studies. Carboxyl quantum dots were supplied by Invitrogen (Qdot® 605 ITK™).

The conjugation reaction between the amino-groups of PLL and carboxyl-groups of quantum dots was carried out as specified by the supplier. Briefly, 4 ml of PLL-BNNTs (50 μg/ml) were mixed with 4 μl of Qdots (8 μM) and 60 μl of 1-ethyl-3-(3-dimethylamino-propyl) carbodiimide (10 mg/ml, EDC, 03450 from Fluka) as activator.

The solution was gently stirred for 90 min at room temperature for optimal conjugation and finally centrifuged (1,000×g, 10 min) to remove large aggregates. Finally, ultracentrifugation (2 cycles at 30,000×g for 30 min at 4° C.) was performed to remove unbound quantum dots and thereby obtain the dispersion of labeled BNNTs (QD-PLL-BNNTs).

### Example 3

### MTT Assay

To evaluate cell viability, MTT (3-(4,5-dimethylthiazole-2-yl)-2,5-diphenyl tetrazolium bromide, M-2128 from Sigma) cell proliferation assays were carried out after 24, 48, and 72 h of incubation with PLL-BNNT modified media, which contained a final concentration of BNNTs equal to 5, 10 and 15 μg/ml. After trypsinization and cell counting with a Bürker chamber, HOBs were plated in 96-well plates. Once the adhesion was verified (after about 6 h from the seeding), cells were incubated with MTT 0.5 mg/ml for 2 h. Then, 100 μl of dimethyl sulfoxide (DMSO, Sigma) were added in each well and absorbance at 550 nm was measured with a VERSAMax microplate reader (Molecular Devices). A reference test (cells cultured in the absence of BNNTs) was carried out as control.

### Example 4

### Intracellular Trackability of Fluorescent BNNTs

The QD-PLL-BNNTs were added to the culture medium in a 1:10 ratio, for a final concentration of PLL-BNNTs equal to 5.0 μg/ml. Cell internalization studies were carried out with fluorescence microscopy after 6 h of incubation (60,000 cells in a 24-well plate). The lysosome tracking assay was carried out on HOBs incubated with Lyso Tracker dye (Invitrogen). This is a fluorescent acidotropic dye for labeling acid organelles in live cells. The fluorescent dye accumulates in cellular compartments characterized by a low pH. For these studies, cells were incubated 2 h in a culture medium containing Lyso tracker in a dilution of 1:2,500 after six h exposure to QD-PLL-BNNTs.

### Example 5

### Analysis by Transmission Electron Microscopy (TEM)

For TEM analysis, HOBs (control) and HOBs treated overnight with CM containing BNNTs at a concentration of 10 μg/ml (HOBs+BNNTs) were used. The cells, after having been removed from the CM, were centrifuged and fixed in a 0.5% w/v gluteraldehyde-4% w/v formaldehyde solution in PBS 0.1M pH 7.2 for 2 h at 4° C. After washing, the samples were post-fixed in 1% w/v OsO₄ PBS 0.1 M pH 7.2 for 1 h, washed and dehydrated with acidified aceton-dimethylacetal (Fluka, Buchs, Switzerland). Finally, the samples were embedded in Epon/Durcupan resin in BEEM capsules #00 (Structure Probe, West Chester, USA) at 56° C. for 48 h. Ultra-thin sections (20-30 nm thick) were obtained with an Ultrotome Nova ultramicrotome (LKB, Bromma, Sweden) equipped with a diamond knife (Diatome, Biel/Bienne, Switzerland). The sections were placed on 200 square mesh nickel grids, counterstained with saturated aqueous uranyl acetate and lead citrate solutions and then observed in a Jeol JEM-I00SX transmission electron microscope.

### Example 6

### Administration of BNNTs Transducers of Ultrasounds (US) into Electrical Stimuli

This study was planned as reported in Table 1 (below). HOBs, either with or without BNNTs internalization, were exposed to ultrasounds and compared to nonexposed controls.

TABLE 1
Planning of experiments

| | HOBs | HOBs | HOBs | HOBs |
|---|---|---|---|---|
| | (sample) | (ctrl1) | (ctrl2) | (ctrl3) |
| BNNTs | X | x | — | — |
| US | X | — | x | — |

US stimulation was carried out according to the scheme: 20 W, for 5 s, 3 times/day for 1 week.

Upon ending the stimulation, the samples, in triplicate for all groups (300,000 cells/flask), were employed for quantitative assays of DNA and bone-specific biomolecules (alkaline phosphatasis and osteocalcin), whereas one sample per each group was used to investigate gene expressions (Runx2, AP, osteopontin, Collagen I, osteocalcin genes) by RT-PCR. Moreover, other HOBs samples were cultivated on slides (20,000 cells/slide) for cytochemistry studies (Von Kossa staining for calcium deposits).

### Example 7

### Total RNA and Reverse Transcriptase-Polymerase Chain Reaction (RT-PCR)

Total RNA was isolated from cell cultures (1 sample/group) using High Pure RNA Isolation kit (Roche, Mannheim, Germany) according to the manufacturer's instructions. Extracted RNA was resuspended in water treated with diethylpyrocarbonate (DEPC-water) and RNA concentration was measured by assessing absorbance at 260 nm. Identical amounts of RNA were reverse transcribed into cDNA using the Transcriptor First Strand cDNA Synthesis kit (Roche).

Subsequently, cDNA was amplified by polymerase chain reaction (PCR). PCR conditions and primers utilized for the amplification of Runx2/cbfa-1, alkaline phosphatasis (AP), osteopontin (OPN), collagen type Iα2 (Coll-I), osteocalcin (OCN) and the housekeeping gene GAPDH are reported in Table 2. The PCR products were loaded on a 2.5% agarose gel and stained with ethidium bromide.

TABLE 2
Primer sequences and conditions for RT-PCR

| Gene | Sequence | bp | Cycle |
|---|---|---|---|
| Gapdh | 5′-GCCAAAAGGGTCATCAT | 347 | 25 cycles: |
| | CTCTG-3′ | | 30 sec, 96° C. |
| | 5′-CATGCCAGTGAGCTTCC | | 60 sec, 58° C. |
| | CGT-3′ | | 30 sec, 74° C. |
| Runx2 | 5′-GCCAAAAGGGTCATCAT | 92 | 35 cycles: |
| | CTCTG-3′ | | 30 sec, 94° C. |
| | 5′-CATGCCAGTGAGCTTCC | | 30 sec, 57° C. |
| | CGT-3′ | | 30 sec, 72° C. |
| AP | 5′-GAGATGGACAAGTTCCC | 518 | 35 cycles: |
| | CTT-3′ | | 45 sec, 94° C. |
| | 5′-TTGAAGCTCTTCCAGGT | | 45 sec, 54° C. |
| | GTC-3′ | | 45 sec, 72° C. |
| OPN | 5′-GCCGAGGTGATAGTGTG | 101 | 35 cycles: |
| | GTT-3′ | | 30 sec, 94° C. |
| | 5′-TGAGGTGATGTCCTCGT | | 30 sec, 57° C. |
| | CTG-3′ | | 30 sec, 72° C. |
| Coll-l | 5′-AAGGTCATGCTGGTCTT | 114 | 35 cycles: |
| | GCT-3′ | | 30 sec, 94° C. |
| | 5′-GACCCTGTTCACCTTTT | | 30 sec, 57° C. |
| | CCA-3′ | | 30 sec, 72° C. |
| OCN | 5′-CGCAGCCACCGAGACAC | 400 | 35 cycles: |
| | CAT-3′ | | 45 sec, 94° C. |
| | 5′-AGGGCAAGGGGAAGAGG | | 45 sec, 60° C. |
| | AAAGAAG-3′ | | 45 sec, 72° C. |

### Example 8

### Cell Sample Preparation for Quantitative Analysis

For the following assays (DNA and OCN content), the cell samples were cultured one week in T25 flasks (n=3). Both assays were carried out in cascade on the same samples. Moreover, individual samples were run in triplicate to minimize operator error. Briefly, the culture medium was carefully removed from the cell samples and ddH₂O was added; then, the samples were frozen at −20° C. and thus stored for subsequent assays. To obtain cell lysates, the samples were subjected to 2 freeze/thaw cycles: overnight freezing at −20° C., 10 min thawing at 37° C. in a water bath, and subsequently stirred for 15 s to enable the DNA and the proteins to go into solution.

9. Preliminary Assays

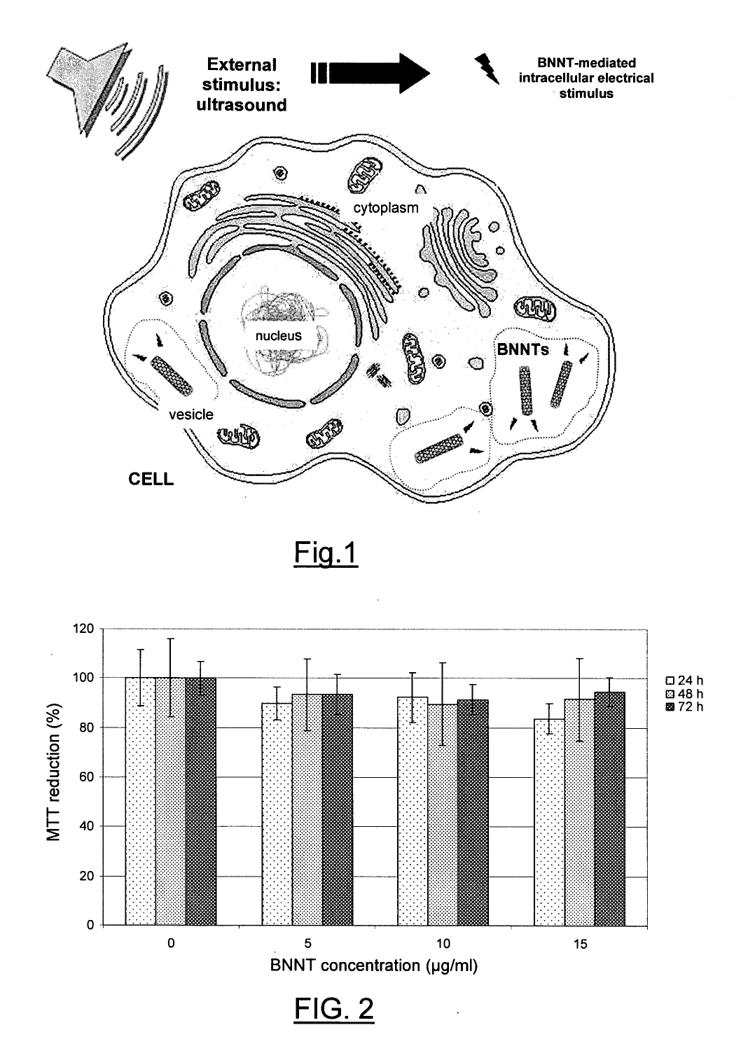

FIG. 1 shows a schematic model reproducing the invention.

9.1. First of all, HOBs were exposed to a BNNTs-containing medium. Stable dispersions of BNNTs in the culture medium were obtained using PLL as dispersion agent. PLL is a cytocompatible polymer with positive amino-terminal groups. After 24, 48, 72 h of incubation with different concentrations of PLL-BNNTs (5, 10 and 15 μg/ml), HOBs viability did not differ from controls (FIG. 2). HOBs did not exhibit a statistically significant decrease in metabolic activity following incubation with PLL-BNNTs at the concentrations used (in all cases, p>0.05 with respect to the controls). Subsequent experiments were carried out using the 10 μg/ml dose.

9.2. The fluorescence assay with fluorescent BNNTs highlighted that BNNTs internalization in human osteoblasts occurs after 6 h of incubation of the cells with a medium containing BNNTs (FIG. 3).

9.3. BNNTs internalization by HOBs was also confirmed by TEM. TEM analysis highlighted that inorganic nanoparticles having shapes and size compatible with said BNNTs may be detected in cytoplasmic vesicles only in samples of treated cells (FIG. 4). Internalization occurs by endocytosis.

10. Effect of Nanotransducers and US in HOBs Cultures

10.1 DNA Content

Double-stranded DNA (ds-DNA) content in cell lysates was measured using the PicoGreen kit (Molecular Probes, Eugene, Oreg.). The PicoGreen dye binds to ds-DNA and the resulting fluorescence intensity is directly proportional to the ds-DNA concentration in solution. Standard solutions of DNA in ddH₂O at concentrations ranging from 0-6 μg/mL were prepared and 50 μl of standard or sample to be measured was loaded for quantification in a 96-well plate. Working buffer and PicoGreen dye solution were prepared according to the manufacturer's instructions and 100 and 150 μl/well added, respectively. After a 10 min incubation in the dark at room temperature, fluorescence intensity was measured on a plate reader (Victor³, PerkinElmer Inc., MA, USA) using an excitation wavelength of 485 nm and an emission wavelength of 535 nm. Cell number was calculated by considering the following relationship: 1 human diploid cell=7.18 μg DNA.

10.2. Osteocalcin (OCN) Production

Osteocalcin (γ-carboxyglutamic acid) is a highly specific bone protein, synthesized by osteoblasts, which may be considered as a metabolic activity marker specific of these cells. OCN was measured in the same lysates employed to assess ALP activity and DNA content, using an immunoenzymatic ELISA N-MID Osteocalcin kit (Cobas, Roche, Indianapolis, Ind., USA), according to the manufacturer's indications.

10.3. Cytochemical Analysis for the Calcium Matrix

to HOBs maturation was investigated with Von Kossa staining, demonstrating the deposition of a hydroxyapatite matrix. HOBs grown on slides were fixed with 1% formalin (Bio-Optica) for 10 min at 4° C. and stained for 15 min with 1% silver nitrate (Fluka, Milwaukee, Wis., USA and Sigma). Staining was developed by incubating the cells with 0.5% pyrogallol (Fluka) and then stirring them 5 times with 5% of sodium thiosulfate (Fulka) for 5 min. Finally, the cells were counterstained with 0.1% of nuclear fast red dye (Fluka). The samples were dehydrated and mounted with DPX (Fluka). The mineral deposit was evaluated as black granules by using optical light microscopy.

10.4. Statistical Analysis Method

Analysis of the data was performed by analysis of variance (ANOVA) followed by Student's t-test to test for significance, which was set at 5%. MTT tests were performed in esaplicate; all the other assays in triplicate. In all cases, three independent experiments were carried out. Results are presented as mean value±standard error of the mean (SEM).

10.5. Results

HOB cells (HOBs) were treated at combined BNNTs+US stimulation for a week as above-indicated.

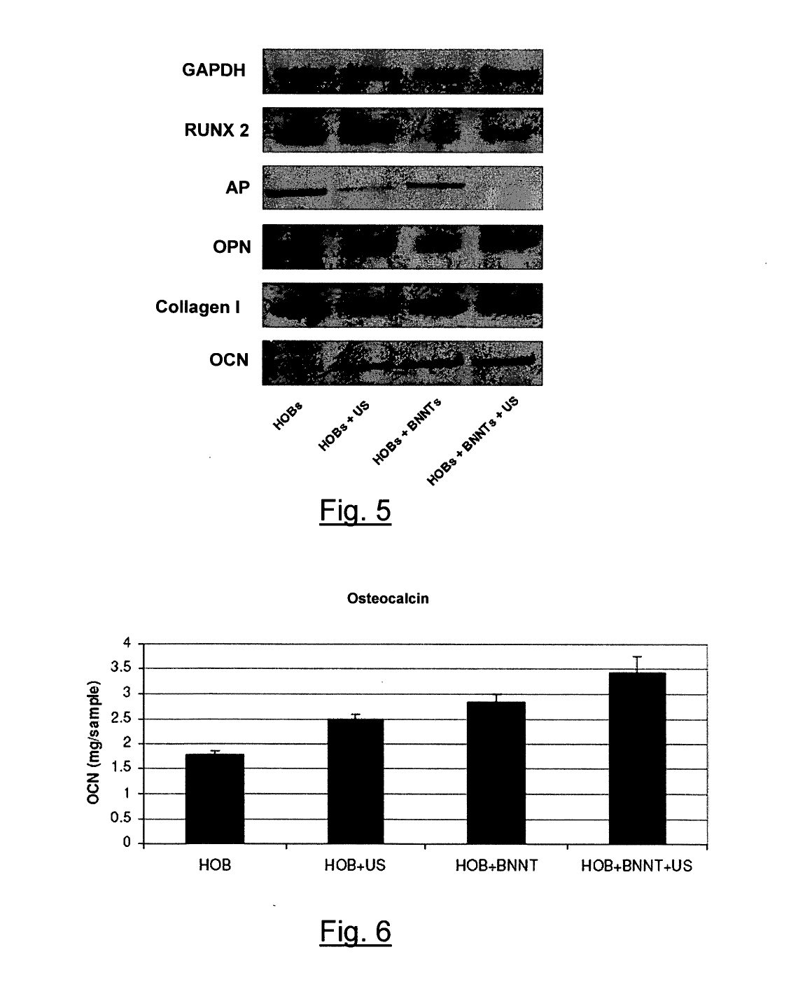

The expression of genes indicating HOBs maturation, specifically of early (Runx2, Coll I, AP e OPN) and late (OPN, OCN) differentiation, was investigated by RT-PCR. OPN has a bimodal expression, which can be early in the proliferative stage and late at the start of mineralization. The results are reported in FIG. 5. Amplification by RT-PCR highlighted the effect of electrical stimulation on HOBs differentiation as a result of a single (BNNTs or US) or combined (BNNTs and US) treatment. In particular, Runx2 was found to be depressed by BNNTs, whereas OPN expression levels were stimulated by US. Coll I expression is unvaried, whereas AP and OCN expressions are synergistically influenced by combined BNNTs+US treatments. In particular, AP is more depressed by US than by BNNTs, and the combined treatment (BNNTs+US) further reduces its expression. Conversely, OCN expression is stimulated by both individual treatments, yet reaches the maximum levels following a combined treatment (BNNTs+US).

In addition, the synthesis of OCN, a protein highly specific of the late stage of osteoblasts, successive to OCN gene activation, was quantified (FIG. 6). OCN synthesis in HOBs is slightly increased by a single treatment with US and highly increased by a single treatment with BNNTs. However, OCN production in cells as a result of a combined BNNTs+US treatment was maximum and highlighted a synergistic effect.

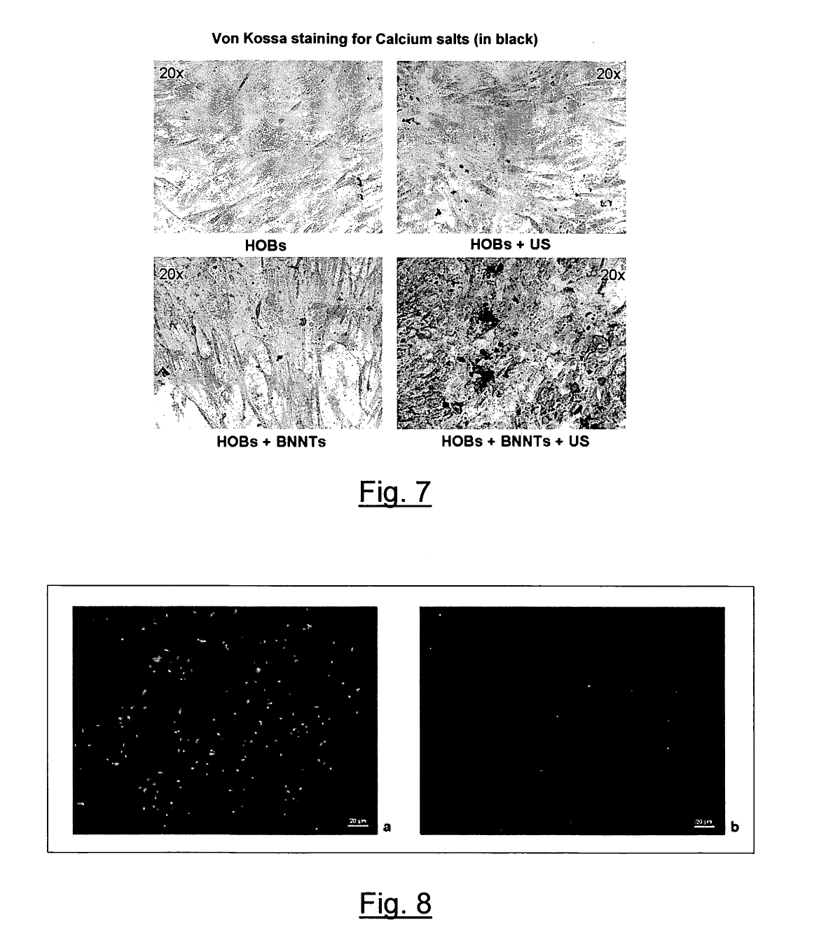

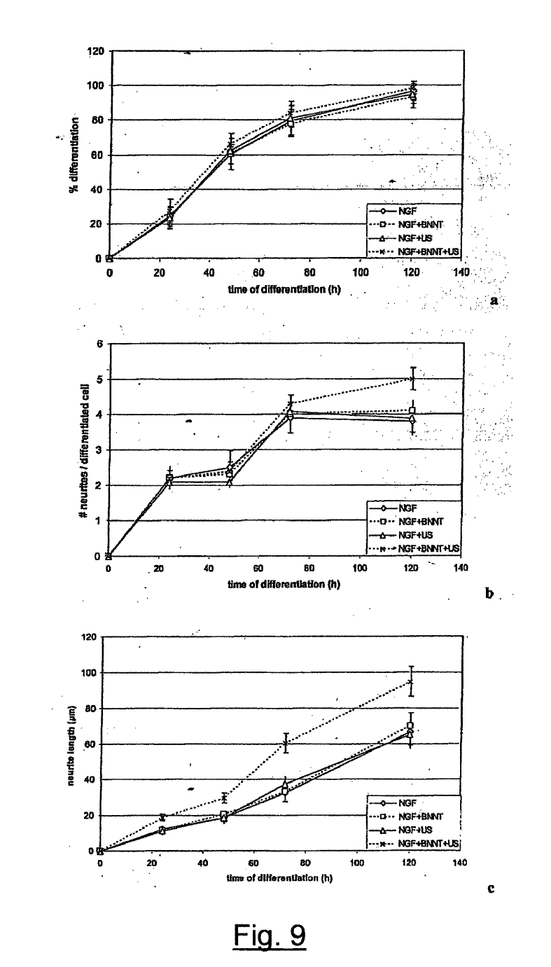

Finally, the cytochemical analysis with von Kossa staining onto slide revealed the highest synthesis of calcium deposits (black staining) in samples subjected to combined treatment (FIG. 7). The depositing of the calcium matrix occurs in mature osteoblasts as a late maturation phase.

Conclusions

Our remarkable results highlight that this combined treatment influences the cell system in a specific manner which is not merely due to the sum of the individual stimuli. In particular, in samples treated with BNNTs+US, downregulation of early genes (Runx2, AP) and upregulation of late genes (OPN and OCN) were observed. OCN is a marker highly specific of the late phase of osteogenesis, which indicates differentiation of mature osteoblasts and undergoing mineralization. Current OCN production was quantitated and found to be of 27 fg/cell. Finally, induction of calcium deposit was demonstrated by cytochemistry. Therefore, it can be concluded that BNNTs act as intracellular nanotransducers, promoting maturation of osteoblasts in vitro following ultrasonic stimulation.

### Example 11

### Preparation of Glycol-Chitosan Polymers Comprising Boron Nitride Nanotubes (GC-BNNT)

BNNTs were purchased from the Nano and Ceramic Materials Research Center, Wuhan Institute of Technology, China. Details of sample purity and composition (provided by the supplier) included: yield >80%, boron nitride 98.5% wt.

The polymer used for BNNTs dispersion and stabilization was Glycol chitosan (G-chitosan 81339, purchased from Sigma with the code G7753). All experiments were carried out in phosphate buffered solution (PBS). Briefly, BNNTs (5 mg) were mixed in 10 ml of a 0.1% G-chitosan solution in a polystyrene tube. The samples were sonicated for 12 h (by a Bransonic sonicator 2510) using a power of 20 W, thereby obtaining a stable G-chitosan-BNNT dispersion in which the BNTT nanotube walls have a non-covalently bound coating of G-chitosan. The dispersion thus obtained was characterized by spectrophotometric analysis, using a LIBRA S12 spectrophotometer UV/Vis/NIR (Biochrom). Microphotographs of the dispersion of BNNTs were obtained with a FEI 200 FIB microscope and with a Zeiss 902 TEM.

### Example 12

### MTT Assay with PC12 Cells Incubated with a Medium Modified with Preparation of Glycol-Chitosan Polymers Comprising Boron Nitride Nanotubes (GC-BNNT)

For viability testing, MTT (3-(4,5-dimethylthiazole-2-yl)-2,5-diphenyltetrazolium bromide, M-2128 from Sigma) cell proliferation assays were carried out on PC12 cells (ATCC CRL-1721) after 24, 48, 72 h of incubation with a medium modified with a dispersion of GC-BNNT, containing a final concentration of BNNT comprised between 0 and 100 μg/ml. After trypsinization and cell count with Bürker chamber, HOBs were seeded in six 96-well plates. Once the adhesion was verified (after about 6 h from the seeding), cells were incubated with MTT 0.5 mg/ml for 2 h. Then, 100 μl of dimethylsulfoxide (DMSO, Sigma) were added into each well and absorbance at 550 nm was measured with a VERSAMax microplate reader (Molecular Devices). A reference control test (k-; cells cultured in the absence of BNNT) was carried out.

### Example 13

### Internalization Test of Preparations of Glycol-Chitosan Polymers Comprising Boron Nitride Nanotubes (GC-BNNT) in PC12 Cells

Studies on internalization of GC-BNNT dispersion were carried out on PC12 cell lines (ATCC CRL-1721). PC12 cells were cultured in modified Dulbecco medium with 10% horse serum and 5% fetal bovine serum, 100 IU/ml penicillin, 100 μg/ml streptomycin and 2 mM L-glutamine. Cells were maintained at 37° C. (i.e., 95% air/5% CO₂).

Nanotube (GC-BNNT) internalization was analyzed by TEM (transmission electron microscopy). PC12 cells were cultured to a concentration of 2×10⁶ cells/T25 plate. After adhesion, the cells were incubated with GC-BNNT-containing CM, to the final concentration of 5 μg/ml for 12 h. The cells, after having been removed from the CM, were centrifuged and fixed with a 0.5% w/v gluteraldeide-4% w/v formaldeide solution in PBS 0.1M pH 7.2 for 2 h at 4° C. After washing, the samples were post-fixed in 1% w/v OsO₄ PBS 0.1 M pH 7.2 for 1 h, washed and dehydrated with acidified to aceton-dimethylacetal (Fluka, Buchs, Switzerland). Finally, the samples were embedded in Epon/Durcupan resin in BEEM #00 capsules (Structure Probe, West Chester, USA) at 56° C. for 48 h. Ultra-thin sections (20-30 nm thick) were obtained con Ultrotome Nova ultramicrotome (LKB, Bromma, Sweden) equipped with a diamond knife (Diatome, Biel/Bienne, Switzerland). The sections were placed on 200 square mesh nickel grids counterstained with saturated aqueous uranyl acetate and lead citrate solutions and then observed in a Zeiss 902 transmission electron microscope.

### Example 14

### PC12 Cell Stimulation Experiments

PC12 cells were plated and kept in standard culture conditions for 24 h. Then, standard CM was replaced with differentiating medium comprising 2% fetal bovine serum, 100 IU/ml penicillin, 100 μg/ml streptomycin, 2 mM L-glutamine and NGF (purchased from SIGMA) at a concentration of 60 ng/ml. The cells thus prepared were utilized in four experiments carried out in parallel: 1) cells cultured in differentiating medium without ultrasound (US) stimulation 2) cells cultured in differentiating medium with US stimulation 3) cells cultured in the presence of a medium containing GC-BNNT (5 μg/ml) 4) cells cultured in the presence of a medium containing GC-BNNT (5 μg/ml) with US stimulation. A stimulation of 20 W, 40 kHz, for 5 s, 4 times/day for 5 days was used, utilizing a Bransonic 2510 sonicator.

For each of the four experiments, more than 50 cells were labeled with 2 μM calcein and analyzed by digital images for evaluation of differentiation, neurite length, number of neurites per cell.

### Example 15

### Results Obtained from PC12 Cells Stimulation Experiments

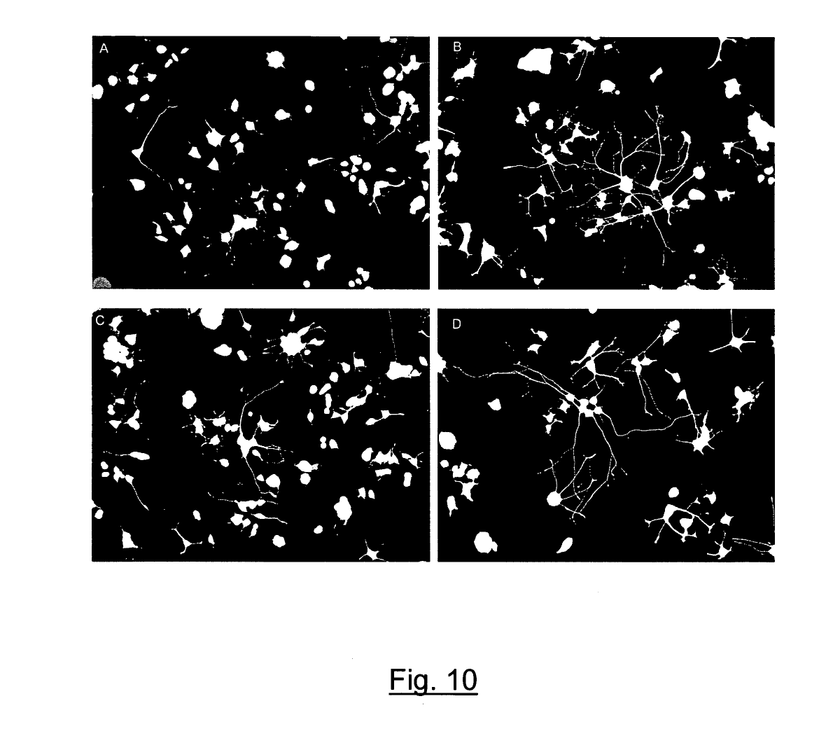

PC12 cells were treated with a combined stimulation of GC-BNNTs and ultrasounds (US) for 5 days, as indicated above. No significant differences in cell differentiation were detected in each of the four experiments (FIG. 10a), all cells reached 95% differentiation without any significant statistic difference (p>0.05). FIG. 6c shows that the group of cells incubated with GC-BNNT and stimulated with ultrasounds has an average neurite length greater than the other groups. These results clearly show that ultrasound stimulation of cells incubated in the presence of polymers comprising nanotubes determines a very pronounced growth of neurites in neural cells.

1. A method for using piezoelectric nanotransducers in an in vivo treatment of cell stimulation through electrical stimulation, comprising:
localizing the piezoelectric nanotransducers in a target site;
inducing an electrical stimulus in the target site through external stimulation of the piezoelectric nanotransducers by ultrasonic waves.

2. The piezoelectric method according to claim 1, further comprising:
coating the piezoelectric nanotransducers with pharmaceutically acceptable polymers, thus providing biocompatible piezoelectric nanotransducers.

3. The method according to claim 1, further comprising:
functionalizing the piezoelectric nanotransducers with specific ligands having affinity for the target site and/or with marker molecules that allow tracking thereof.

4. The method according to claim 1, wherein the piezoelectric nanotransducers are boron nitride nanotubes.

5. The method according to claim 1, wherein the piezoelectric nanotransducers are dispersed in a non-aggregated form into a stable suspension.

6. The method according to claim 1, wherein said treatment is a regenerative or reconstructive treatment of tissues, a pain treatment or a healing treatment of damaged tissues.

7. The method according to claim 1, wherein the localization of the piezoelectric nanotransducers occurs via cell internalization.

8. The method according to claim 1, wherein the target site is selected among muscle cells, myoblasts, neural cells, myocardial cells, osteoblasts, osteoclasts, stem cells, sensory cells such as inner ear hair cells, rods and cones of the retina, cells of taste, cells of touch and cells of smell.

9. A pharmaceutical preparation comprising piezoelectric nanotubes capable of being stimulated by an ultrasonic remote field and a pharmaceutically acceptable excipient for use in a treatment of electrotherapy.

10. The pharmaceutical preparation according to claim 9, said preparation being in liquid form, wherein said piezoelectric nanotubes are dispersed in a non-aggregated form, the preparation further comprising a biocompatible polymer as dispersing agent.

11. The pharmaceutical preparation according to claim 9, wherein the piezoelectric nanotubes are encapsulated in lipid or phospholipid microbubbles containing a harmless gas.

12. The pharmaceutical preparation according to claim 9, wherein the piezoelectric nanotubes biocompatible piezoelectric nanotubes coated with pharmaceutically acceptable polymers and/or functionalized with specific ligands having affinity for a target site and/or marker molecules that allow tracking thereof.

13. The pharmaceutical preparation according to claim 9, wherein the nanotubes are boron nitride nanotubes.

14. A polymeric or ceramic support for cell growth or tissue engineering, in vitro or in vivo, comprising piezoelectric nanotransducers capable of producing an electrical stimulus as a result of external stimulation with ultrasounds.

15. The support according to claim 14, wherein the piezoelectric nanotransducers are biocompatible piezoelectric nanotransducers coated with pharmaceutically acceptable polymers and/or functionalized with specific ligands and/or marker molecules that allow tracking thereof.

16. The support according to claim 14, wherein the piezoelectric transducers are boron nitride nanotubes.

17. A method for preparing supports according to claim 14, comprising:
dispersing the piezoelectric nanotransducers into a solution, dispersion or emulsion containing a polymer or its monomers; and
removing liquid medium from the solution, dispersion or emulsion, thus obtaining a solid or semi-solid matrix containing the piezoelectric nanotransducers.

18. A method in vitro for cell stimulation via electrical stimulation, comprising:
dispersing piezoelectric nanotransducers in culture medium or cell growth supports;
incubating cells in said culture medium or growth supports; and
inducing an electrical stimulus through a stimulation of the piezoelectric nanotransducers by an ultrasonic field external to the culture medium or cell growth supports.

19. The method according to claim 18, wherein the cell growth supports are polymeric or ceramic scaffolds for tissue engineering, implant or adhesion substrates.

20. The method according to claim 18, wherein the piezoelectric nanotransducers are made biocompatible by coating with pharmaceutically acceptable polymers and/or functionalized with specific ligands having affinity for a target cell and/or marker molecules that allow tracking thereof.

21. The method according to claim 18, wherein the piezoelectric nanotransducers are boron nitride nanotubes.
